(19) United States
(12) Patent Application Publication
(10) Pub. No.: US 2021/0322733 A1
(43) Pub. Date: Oct. 21, 2021

(54) APPARATUS AND METHOD FOR TREATING GASTROINTESTINAL TUMORS

(71) Applicant: BRAXX BIOTECH CO., LTD., Taipei (TW)

(72) Inventors: Kai-Lin YANG, Taipei (TW); Jeng-Yu CHOU, Yilan (TW)

(21) Appl. No.: 17/294,671

(22) PCT Filed: Apr. 13, 2020
(86) PCT No.: PCT/CN2020/084406

§ 371 Date: May 17, 2021

Related U.S. Application Data
(60) Provisional application No. 62/837,738, filed on Apr. 24, 2019.

(51) Int. Cl. A61M 25/10; A61N 5/10; A61M 25/00; A61M 5/00

(52) U.S. Cl. CPC A61M 25/1011; A61N 5/1077; A61M 25/0026; A61M 2205/3379; A61M 5/007; A61N 2005/1087; A61M 2210/105; A61M 25/10186

(57) ABSTRACT

Disclosed herein is an apparatus for use with external beam radiotherapy (EBRT). According to embodiments of the present disclosure, the apparatus comprises a catheter and a plurality of compliant balloons extended outside and along the axial direction of the catheter, wherein the catheter comprises a plurality of communicating conduits, each of which is in air or fluid communication with at least one of the plurality of compliant balloons. Also disclosed herein are methods of treating gastrointestinal tumors in a subject with the aid of the present apparatus.

### CROSS-REFERENCE TO RELATED APPLICATIONS

This application relates to and claims the benefit of U.S. Provisional Application No. 62/837,738, filed Apr. 24, 2019; the content of the application is incorporated herein by reference in its entirety.

### BACKGROUND OF THE INVENTION

### 1. Field of the Invention

The present disclosure in general relates to the field of tumor treatment. More particularly, the present disclosure relates to an apparatus for use with external beam radiotherapy (EBRT) thereby treating gastrointestinal tumors.

### 2. Description of Related Art

Gastrointestinal tumor is a disease involving abnormal cell growth that occurs in the gastrointestinal tract (GI tract) and accessory organs of digestion, for example, the esophagus, stomach, biliary system, pancreas, small intestine, large intestine, rectum, and anus, in which the esophageal, stomach, and pancreatic tumors are respectively the sixth, the fourth, and the fifth leading cause of cancer-related mortality. The symptoms of gastrointestinal tumors vary with the organs or tissues affected. For example, symptoms associated with the esophageal tumor include, difficulty in swallowing, chest pain, coughing, and hoarseness; while symptoms associated with the stomach tumor include, vomiting, nausea, abdominal pain, and blood in the stool.

Radiation therapy is one major treatment for gastrointestinal tumors. In general, surgery in combination with radiation therapy and/or chemotherapy is recommended for treating patient with a tumor without spreading beyond the GI tract and lymph nodes. For advanced gastrointestinal tumors, treatment usually involves chemotherapy and radiation therapy. There are two main types of radiation therapy, external beam radiation therapy (EBRT) (i.e., a radiation treatment given from a machine outside the body, for example, X-ray therapy and proton beam therapy (PBT)), and internal radiation therapy (i.e., a radiation treatment given directly inside the body, also known as “brachytherapy”). However, neither the external beam radiation therapy nor the internal radiation therapy provides a satisfactory effect on gastrointestinal tumors due to the limitations of undesired side-effects (such as, nausea, sunburn-like skin reaction, pain or difficulty with swallowing, heart damage, lung damage, and upset stomach) that results from the damage to normal cells and tissues near the treatment area. Thus, it is important to improve the accuracy of the radiation therapy thereby reducing the side-effects.

Particle therapy (e.g., proton therapy) appears to be safer and more effective than conventional radiation therapy. It is well known that the advantage of a particle beam is the physical characteristics of its depth-dose curve, with a dose peak (Bragg peak) at a well-defined depth in tissue. For relatively shallow tumors, unlike the photon depth-dose curve showing an exponentially decreasing energy deposition with increasing depth in tissue, the Bragg peak allows for rapid fall-off of the radiation dose at the end of the range and a sharp lateral dose fall-off with the maximum energy deposition for each particle beam in the target region and almost no energy around it. Therefore, particle therapy effectively allows the delivery of high-radiation doses to tumor cells and very low or zero doses to the normal cells, which is recognized as an ideal therapy modality for treatment of malignant diseases, especially for organs at risk (OARs) with less toxicity. However, the precision of particle therapy of tumors situated in thorax and abdominal region (e.g. esophagus) is strongly affected by the body conformation, internal organs characteristics and target motion. These negative influence requires advanced techniques of tumor position monitoring and irradiation.

Several protective spacers configured to protect normal tissues adjacent to tumors from radiation injuries have been developed so as to address the side-effect issue of radiation therapy. One of the potential spacers is balloon catheter, which spaces the tumor away from normal tissues adjacent thereto via inflating one or more balloons against the tumor. Nevertheless, these balloon catheters are rough and imprecise which could only compatible with conventional radiation therapy but couldn't meet the precision requirement of particle therapy. It is also reported that the balloon catheter would cause a tearing injury of the tract as a result of over-inflation of the spacer balloon. Further, the dead space between balloons also diminishes the protective effect of balloon catheters.

In view of the foregoing, there exists in the related art a need for an improved apparatus for assisting the performance of radiation therapy thereby improving the accuracy and safety thereof.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to an apparatus for use with an EBRT to treat a gastrointestinal tumor in a subject. The apparatus comprises a catheter and a plurality of compliant balloons extended outside and along the axial direction of the catheter. According to embodiments of the present disclosure, the catheter comprises a plurality of communicating conduits, and each of the plurality of communicating conduits is in air or fluid communication with at least one (e.g., one, two, three, four, five, or more) of the plurality of compliant balloons. In one example, each of the plurality of communicating conduits is in air or fluid communication with one compliant balloon. In another example, each of the plurality of communicating conduits is in air or fluid communication with more than one compliant balloons, e.g., two, three, or four compliant balloons. As could be appreciated, the communication of the communicating conduit and the compliant balloon may vary in accordance with desired purposes; for example, the catheter may comprise four communicating conduits (i.e., a first to a fourth communicating conduits) and ten compliant balloons (i.e., compliant balloon numbers 1 to 10), in which the first communicating conduits is in communication with one compliant balloon (e.g., compliant balloon number 1), the second communicating conduits is in communication with two compliant balloons (e.g., compliant balloon numbers 2 and 3), and the third and the fourth communicating conduits are respectively in communication with three and four compliant balloons (e.g., compliant balloon numbers 4 to 6, and compliant balloon numbers 7 to 10).

The present apparatus is characterized in that once it enters the gastrointestinal tract of a subject, each of the plurality of compliant balloons is configured to inflate in the axial and radial directions of the compliant balloon thereby conforming to the shape of the gastrointestinal tract of the subject. According to embodiments of the present disclosure, the inflation in the axial direction ensures that there is substantially no dead space present between two adjacent balloons.

Optionally, each of the plurality of compliant balloons independently comprises a supporting structure (e.g., a rib structure) disposed inside and/or outside the compliant balloon.

According to some embodiments of the present disclosure, each of the plurality of compliant balloon is in juxtaposition to its adjacent balloon. In these embodiments, each of the plurality of compliant balloon has a central portion along the axial direction thereof, and a radial portion extending radially outward from the central portion, wherein the axial length of the central portion is equal to or less than the maximum axial length of the radial portion.

According to certain embodiments of the present disclosure, each of the plurality of compliant balloons has two terminal portions and an intermediate portion disposed therebetween, wherein the intermediate portion is relatively thicker than each terminal portion.

Preferably, the apparatus comprises at least three communicating conduits and at least three compliant balloons, and each of the communicating conduits is in air or fluid communication with each of the compliant balloons.

According to certain embodiments of the present disclosure, the catheter further comprises a working conduit disposed adjacent to the plurality of communicating conduits. In these embodiments, the working conduit is configured to receive a medical instrument, an endoscope, a contrast agent, a radionuclide, or a shielding material. Basically, the shielding material is made of a metal, a metal alloy, a polymer, or a combination thereof.

According to one embodiment of the present disclosure, the apparatus further comprises a fluid and/or air supplier, which is operably coupled to the plurality of the communicating conduits, and configured to provide a fluid or an air to the plurality of communicating conduits. According to another embodiment of the present disclosure, the apparatus further comprises a plurality of fluid and/or air suppliers, which are operably coupled to the plurality of the communicating conduits, and configured to independently provide a fluid or an air to the plurality of communicating conduits.

Optionally, the present apparatus further comprises a plurality of valves, which are respectively coupled to the plurality of the communicating conduits, and each valve is configured to independently control the volume of the air or the fluid provided to each communicating conduit so as to alter the inflation volume of each compliant balloon.

Still optionally, the present apparatus further comprises a plurality of indicators, which are respectively coupled to the plurality of the communicating conduits, and each indicator is configured to independently indicate the volume of the air or the fluid provided to each communicating conduit.

According to preferred embodiments of the present disclosure, the apparatus further comprises a cap disposed at the front end of the catheter.

Another aspect of the present disclosure is directed to a radiotherapy system for treating a gastrointestinal tumor in a subject. The radiotherapy system comprises an apparatus in accordance with any embodiment of the present disclosure, and a radiation device for use with the apparatus. According to some embodiments of the present disclosure, the apparatus is configured to space the gastrointestinal tumor away from a normal tissue of the gastrointestinal tract of the subject, and the radiation device is configured to provide an external beam radiation therapy to the gastrointestinal tumor.

Also disclosed herein is a method of treating a gastrointestinal tumor in a subject with the aid of the present apparatus. The method comprises,

(a) inserting the apparatus through the mouth or nose of the subject into the gastrointestinal tract of the subject;

(b) inflating at least one of the compliant balloons so as to keep the gastrointestinal tumor away from a normal tissue of the gastrointestinal tract of the subject;

(c) administering to the gastrointestinal tumor an effective amount of EBRT; and

(d) optionally, adjusting the position of the apparatus via altering the inflation volume of at least one of the compliant balloons to optimize the treatment of the gastrointestinal tumor.

In general, the EBRT may be a photon beam radiation therapy (e.g., X-ray or gamma-ray therapy), or a particle therapy (e.g., proton, neutron or carbon ion therapy). According to some embodiments of the present disclosure, the EBRT is a proton beam therapy (PBT).

The gastrointestinal tumor may be an esophageal tumor, a stomach tumor (also known as gastric tumor), a tumor of bile duct, a gallbladder tumor, a pancreatic tumor, a small intestinal tumor, a colon tumor, a rectal tumor, or an anal tumor. According to one embodiment of the present disclosure, the gastrointestinal tumor is esophageal tumor.

The subject is a mammal; preferably, a human.

The present apparatus with independently inflatable compliant balloons (and supporting structure) is useful in spacing the gastrointestinal tumor away from a normal organ and/or tissue of the gastrointestinal tract of a subject, and eliminating the dead space during radiation therapy (e.g., particle therapy), thereby reducing unnecessary exposure of the normal organ/tissue (e.g., the organ/tissue surrounding the tumor, or the organ at risk (OAR)) to radiation.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A to 1E are respectively the side views and the sectional views of the present apparatus according to one embodiment of the present disclosure.

FIG. 1F is the partial enlargement view of the compliant balloon of the present apparatus according to one embodiment of the present disclosure.

FIG. 1G provides schematic diagrams of the compliant balloon before and after inflating according to another embodiment of the present disclosure.

FIG. 2 is the sectional view of the compliant balloon of the present apparatus according to one embodiment of the present disclosure.

FIG. 3 is a schematic diagram illustrating the inflating state of the present apparatus according to another embodiment of the present disclosure.

FIG. 4 is the sectional view of the present apparatus according to another embodiment of the present disclosure.

FIGS. 5A-5D are schematic diagrams respectively illustrating apparatuses comprising suppliers, valves, indicators and/or a cap according to one embodiment of the present disclosure.

FIG. 6 is a schematic diagram illustrating the present radiotherapy system according to one embodiment of the present disclosure.

FIG. 7 is a schematic diagram illustrating the practical application of present apparatus according to another embodiment of the present disclosure.

FIG. 8 provides schematic diagram illustrating the practical application of present apparatus according to another embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “dead space” as used herein refers to a space present between two compliant balloons when they are in an inflating state. The term “substantially no dead space” means that the amount of the dead space is less than about 10% of the inflation volume of the compliant balloon; preferably, less than 5% the inflation volume of the compliant balloon; more preferably, less than 3% the inflation volume of the compliant balloon; even more preferably, less than 1% of the inflation volume of the compliant balloon.

As used herein, the term “operably coupled” refers to two components (e.g., the fluid and/or air supplier and the communicating conduit of the present apparatus) are in air or fluid communication with each other either directly or indirectly thought other intermediate members or components.

The term “valve” as used herein refers to any flow regulating device or system. For example, the term “valve” can include, without limitation, any device or system that controllably allows, prevents, or inhibits the flow of the air or fluid through a passageway (e.g., the communicating conduit of the present apparatus). The term “valve” can be a pinch valve, rotary valve, stop cock, pressure valve, shuttle valve, mechanical valve, electrical valve, electro-mechanical flow regulator, or a combination thereof.

The term “treat” and “treatment” are used interchangeably and refer to the use of the apparatus of the present invention with EBRT, to alleviate or ameliorate a symptom, a secondary disorder or a condition associated with gastrointestinal tumor in a subject. Symptoms, secondary disorders, and/or conditions associated with gastrointestinal tumor include, but are not limited to, swallowing, chest pain, coughing, hoarseness, vomiting, nausea, abdominal pain, diarrhea, constipation, fatigue, weight loss, and blood in the stool.

As used herein, the term “axial direction” refers to the longitudinal direction of the catheter, the longitudinal direction of the compliant balloon, or the longitudinal direction of the apparatus of the present disclosure.

As used herein, the term “radial direction” refers to a direction orthogonal to the axial direction; i.e., a direction perpendicular to the central axis of the catheter, compliant balloon, or apparatus of the present disclosure. More specifically, the term “radial direction” refers to a direction from the central axis towards the outer or outside periphery of an element (e.g., the compliant balloon of the present apparatus).

The term “circumferential direction” as used herein has its usual meaning and refers to a direction, which is tangent to any circle centered on the axis of rotation. The circumferential direction is perpendicular to both the axial direction and a radial direction.

In the context of the present disclosure, “the front end” of the catheter refers to the end of the catheter or the working conduit that is inserted into the body.

The term “subject” refers to a mammal including the human species that is treatable with the apparatus and/or method of the present invention. The term “subject” is intended to refer to both the male and female gender unless one gender is specifically indicated.

### II. Description of The Invention

The present disclosure aims at providing an apparatus for facilitating radiation therapy thereby improving the accuracy and safety of the radiation therapy. In structure, the apparatus of the present disclosure comprises a plurality of compliant balloons extended along the axial direction of the apparatus, wherein each of the compliant balloons is characterized in having, (1) a supporting structure deposed therein or thereon, and (2) a body of uneven distribution of thickness, in which the body of each compliant balloon is relatively thicker towards the center than to the end portions of the body. As each balloon is made of an elastomeric material, which enables the balloon to expand or inflate in conformity with the shape of the GI tract, and further with the aid of the supporting structure inside/on the balloon and the uneven distribution of thickness in the balloon body, thus, each compliant balloon is in juxtaposition to its neighboring balloon after inflation (i.e., there is substantially no dead space between the two juxtaposed balloons). Accordingly, compared with conventional balloon catheters, which are usually limited by the dead spaces between two neighboring balloons and the adverse effect discussed above (e.g., causing tearing injuries), the present apparatus provides better protection to normal tissues adjacent to the gastrointestinal tumor by reducing unnecessary exposure of the normal tissues to radiation during radiation therapy (e.g., X-ray therapy and PBT). Further, the present apparatus is also advantage in efficiently protecting the organ (such as, heart and lung) from radiation injuries, a common condition occurring during radiation therapy, especially during particle therapy that focuses the energy of particle beam within the tumor while minimizing the damage to nearby healthy tissues and vital organs (e.g., heart and lung).

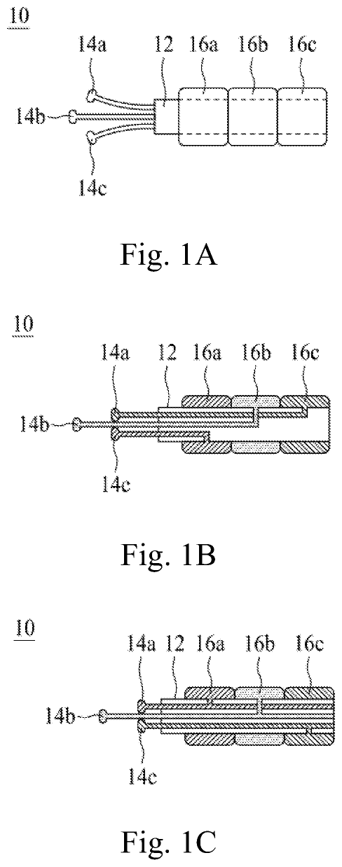

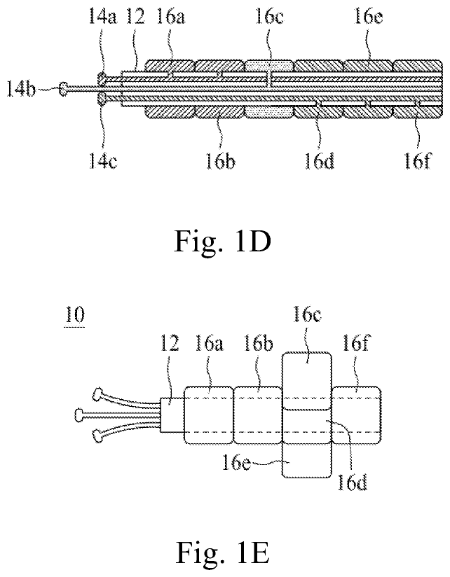

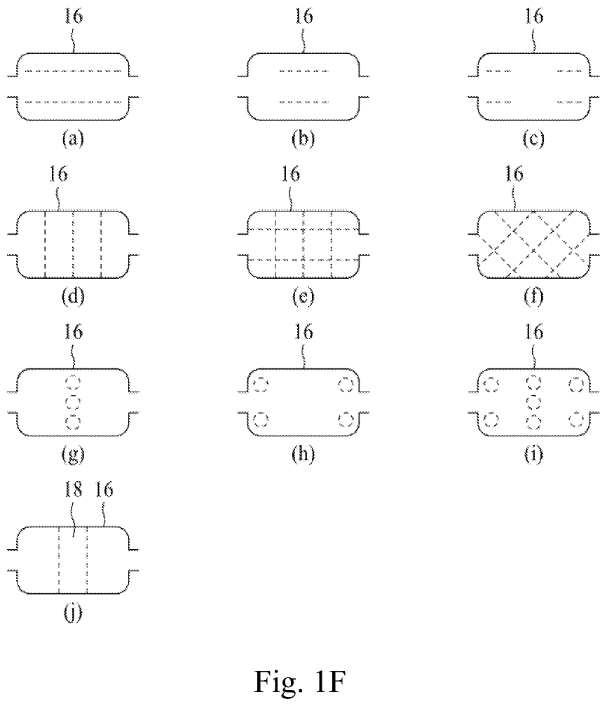

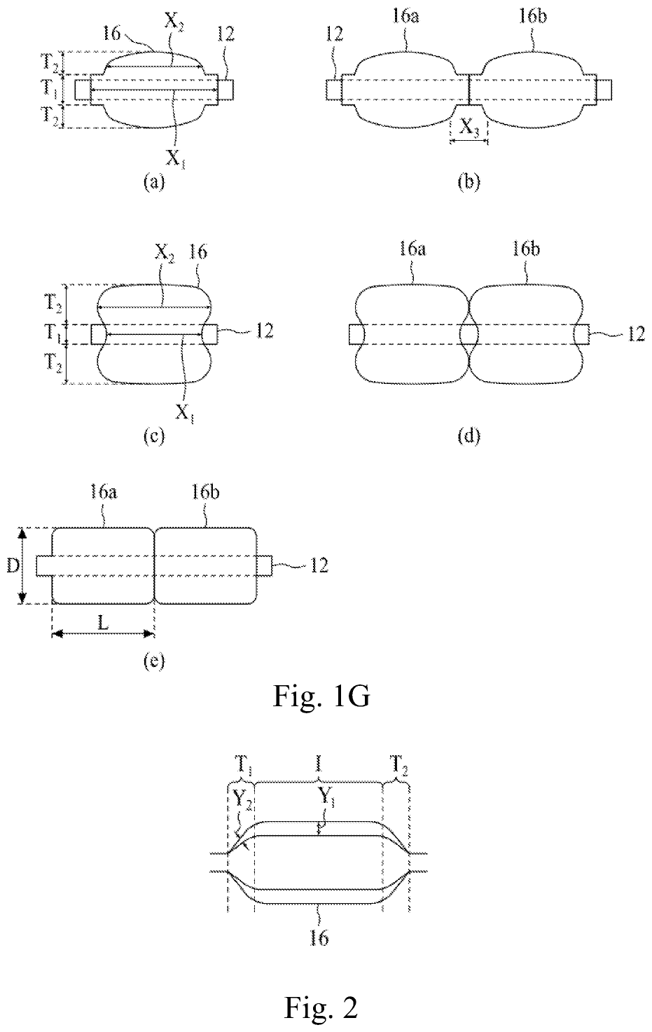

Reference is now made to FIGS. 1A and 1B respectively depicting the side view and the side sectional view of the present apparatus. As illustrated in FIGS. 1A and 1B, the apparatus 10 comprises a catheter 12, and a plurality of compliant balloons 16a, 16b, 16c extended outside and along the axial direction of the catheter 12. Each of the compliant balloons may be secured to the catheter by various methods, for example, glue, envelope, ring and etc. The catheter 12 comprises a plurality of communicating conduits 14a, 14b, 14c, each of which is in air or fluid communication with a corresponding compliant balloon (i.e., the communicating conduit 14a is in air or fluid communication with the compliant balloon 16c, the communicating conduit 14b is in air or fluid communication with the compliant balloon 16b, and the communicating conduit 14c is in air or fluid communication with the compliant balloon 16a). For better understanding, each communicating conduit and the compliant balloon(s) that is/are in communication therewith are marked by the same symbol in FIG. 1. As would be appreciated, the numbers of the compliant balloon and the communicating conduit in communication therewith may vary with desired purposes. According to some preferred embodiments of the present disclosure, the present apparatus comprises at least three (e.g., 3, 4, 5, 6, 7, 8, 9, 10 or more) compliant balloons and at least three (e.g., 3, 4, 5, 6, 7, 8, 9, 10 or more) communicating conduits, in which each compliant balloon is connected to and in communication with a corresponding communicating conduit. In addition, the compliant balloons may have the same or different lengths. For example, the present apparatus may comprise six compliant balloons, three of which are independently about 0.5-1.5 cm (e.g., 0.5, 0.6, 0.7, 0.8, 0.9, 1.0, 1.1, 1.2, 1.3, 1.4, or 1.5 cm) in length, while the other three are independently about 1.5-2.5 cm (e.g., 1.5, 1.6, 1.7, 1.8, 1.9, 2.0, 2.1, 2.2, 2.3, 2.4, or 2.5 cm) in length. Alternatively, the present device may comprise eight compliant balloons, two of which are independently about 2.5-3.5 cm (e.g., 2.5, 2.6, 2.7, 2.8, 2.9, 3.0, 3.1, 3.2, 3.3, 3.4, or 3.5 cm) in length, three of which are about 1.5-2.5 cm (e.g., 1.5, 1.6, 1.7, 1.8, 1.9, 2.0, 2.1, 2.2, 2.3, 2.4, or 2.5 cm) in length, while the other three of which are independently about 0.5-1.5 cm (e.g., 0.5, 0.6, 0.7, 0.8, 0.9, 1.0, 1.1, 1.2, 1.3, 1.4, or 1.5 cm) in length.

According to one embodiment of the present disclosure, the apparatus 10 comprises three communicating conduits and three compliant balloons, in which each compliant balloon is connected to and in air communication with a corresponding communicating conduit. According to another embodiment of the present disclosure, the apparatus 10 comprises three communicating conduits and three compliant balloons, in which each compliant balloon is connected to and in fluid communication (e.g., a contrast agent) with a corresponding communicating conduit.

FIG. 1C provides an alternative embodiment of the present apparatus 10, the configuration of which is quite similar with that of FIG. 1B, except the communicating conduits 16A, 16b, 16c extend along the axial direction of the apparatus.

According to alternative embodiments of the present disclosure, each of the communicating conduits is in air or fluid communication with one or more (e.g., one, two, three, four, five, six, seven, eight, nine, ten, or more) compliant balloons. Reference is now made to FIG. 1D, in which the apparatus comprises three communicating conduits 14a, 14b, 14c. Instead of individually in communication with one compliant balloon as depicted in FIGS. 1A and 1B, the communicating conduits 14a, 14b, 14c are respectively in communication with two, one, and three compliant balloons. According to the schematic diagram of FIG. 1D, the communicating conduit 14a is in communication with the compliant balloons 16a, 16b, the communicating conduit 14b is in communication with the compliant balloon 16c, and the communicating conduit 14c is in communication with the compliant balloons 16d, 16e, 16f. In this manner, the inflation volumes of different compliant balloons (e.g., compliant balloons 16d, 16e, 160 may be simultaneously controlled by one communicating conduit (e.g., communicating conduit 14c).

FIG. 1E provides another alternative embodiment of the present apparatus 10. Compared to the arrangement of FIG. 1A, in which each compliant balloon extends along the axial direction of the catheter, the alternative apparatus 10 is characterized in having three compliant balloons 16c, 16d, 16e respectively extend along the circumferential direction of the catheter 12. In this embodiment, each of the compliant balloons 16c, 16d, 16e may be in air or fluid communication with a corresponding communicating conduits, and accordingly, the inflation volume of each compliant balloon 16c, 16d, 16e is independently controlled by each communicating conduit. Alternatively, the compliant balloons 16c, 16d, 16e may in air or fluid communication with each other, and the inflation volumes thereof are controlled by one communicating conduit.

According to certain embodiments of the present disclosure, when the apparatus enters the gastrointestinal tract of a subject, each compliant balloons is configured to inflate in the axial and radial directions of the compliant balloon thereby conforming to the shape of the gastrointestinal tract of the subject. Specifically, the inflation of the compliant balloon in the axial direction thereof (i.e., inflating alone the axial direction of the apparatus and the compliant balloon) ensures that there is substantially no dead space present between two adjacent balloons, and the inflation of the compliant balloon in the radial direction thereof (i.e., inflating radially outward from the axis of the compliant balloon) efficiently spaces the normal organs and/or tissues of the gastrointestinal tract away from the gastrointestinal tumor, so as to provide a protection to normal organs and/or tissues adjacent to the gastrointestinal tumor during radiation therapy.

According to some alternative embodiments of the present disclosure, each compliant balloon of the present apparatus is made from a single envelope (preferably, an envelope made of an elastomeric material), which has a plurality of axially-spaced annular apertures coupled thereon and/or therein. The plurality of axially-spaced annular apertures are disposed along the axial direction of the catheter thereby dividing the envelope into several independent space. In these embodiments, each space is in air or fluid communication with a communicating conduit, which controls the inflation volume of the independent space.

As indicated above, the compliant balloon of the present disclosure is characterized in having a supporting structure in/on the balloon. The supporting structure may be independently formed and placed within the balloon. Alternatively, it may be integrally formed on the body of the balloon. Reference is now made to FIG. 1F, where partial enlargement views of various configurations of the supporting structures are depicted. In general, the configuration and/or distribution of the supporting structures may vary with desired purposes. For example, the supporting structure may be in the form of a plurality of ribs independently extending along the lateral (or axial) or longitudinal orientation of the compliant balloon 16, and may be disposed at some parts of the balloon, such as at the center portion, or at the terminal portions of the compliant balloon 16, as illustrated in Panels (a)-(d) of FIG. 1F. Alternatively, the ribs may be arranged symmetrically or non-symmetrically, or are in cross configuration with each other at a predetermined angle (e.g., 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170 or 175 degrees) as illustrated in Panels (e)-(f) of FIG. 1F. Still alternatively, the supporting structure may be in the form of a plurality of circular structures disposed at the intermediate portion and/or the terminal portions of the compliant balloon 16, as illustrated in Panels (g)-(i) of FIG. 1F. The above description is merely to exemplify the configuration and/or distribution of the supporting structure; it should be understood that the scope of the present disclosure is not limited thereto. For example, the supporting structure may be in the form of a band structure 18 disposed at the intermediate portion of the compliant balloon 16 as depicted in Panel (j) of FIG. 1F. The inclusion of the supporting structure ensures that the compliant balloons would inflate from the terminal portion to the intermediate portion when the communicating conduits are starting to fill with fluid or air.

FIG. 1G illustrates the configuration of the compliant balloon 16 in accordance with two embodiments of the present disclosure. As depicted in FIG. 1G, the compliant balloon 16 comprises a central portion T₁ along the axial direction of the compliant balloon 16, and a radial portion T₂ extending radially outward from the central portion T₁. Specifically, the compliant balloon 16 may be secured to the catheter 12 in the configuration as depicted in Panel (a) of FIG. 1G, in which the length of the central portion T₁ (i.e., X₁) is greater than the average length of the radial portion T₂ (i.e., X₂) (i.e., X₁>X₂). In this case, two compliant balloons 16a, 16b are separated by a distance X₃ before inflation (See Panel (b) of FIG. 1G), in which X₃ may be 0.01 to 1.0 cm, for example, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0; preferably, X₃ is 0.1 to 0.8 cm. Alternatively, the compliant balloon 16 may be secured to the catheter 12 in the configuration as depicted in Panel (c) of FIG. 1G, in which the length of the central portion T₁ (i.e., X₁) is less than the maximal length of the radial portion T₂ (i.e., X₂) (i.e., X₂>X₁). Thus, two compliant balloons 16a, 16b are independently in juxtaposition to each other before inflation as illustrated in Panel (d) of FIG. 1G. Still alternatively, the length of the central portion T₁ (i.e., X₁) may be equal to the maximal length of the radial portion T₂ (i.e., X₂) (i.e., X₂=X₁). In these embodiments, there is substantially no dead space between the compliant balloons 16a, 16b after inflation (Panel (E) of FIG. 1G). According to one specific example, the length of the central portion T₁ (i.e., X₁) is equal to or less than the maximal length of the radial portion T₂ (i.e., X₂) (i.e., X₂≥X₁). Preferably, after inflation, the diameter (D) of each compliant balloon is equal to or less than five times of the length (L) of the compliant balloon (i.e., D≤5×L) (FIG. 1G).

Another characteristic of the present compliant balloon lies in having a balloon body of non-uniform thickness. Reference is now made to FIG. 2, which is the side sectional view of the compliant balloon 16. For illustration purpose, the compliant balloon 16 is depicted as having three portions: a first terminal portion T₁; a second terminal portion T₂; and an intermediate portion I, which is the portion between the first and second terminal portions T₁ and T₂. The average thickness of the intermediate portion I (Y₁) in general, is greater than the average thickness of each terminal portions T₁, T₂ (Y₂). The difference in thickness between the intermediate portion I and the terminal portions T₁, T₂ ensures that the compliant balloon would be uniformly inflated when the air or fluid is provided thereto.

Additionally or alternatively, the balloon body of the compliant balloon 16 may have non-uniform diameter, in which the average diameter of the intermediate portion is less than that of the terminal portions. In this case, the compliant balloon would inflate from the terminal portion to the intermediate portion when the fluid or air is provided to the communicating conduit.

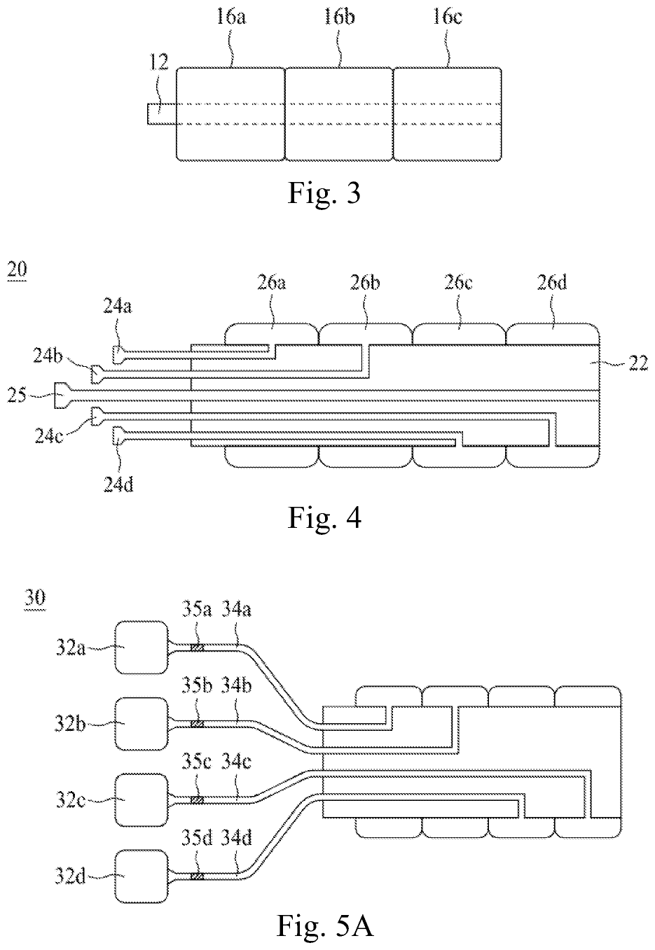

Another feature of the present apparatus lies in the arrangement of the plurality of the compliant balloons. According to certain embodiments of the present disclosure, after inflation, each compliant balloon is in juxtaposition to its neighboring balloon; accordingly, there is minimum or substantially zero dead space between the two juxtaposed balloons as schematically illustrated in FIG. 3. Accordingly, once the compliant balloons are inflated to form a substantially continuous configuration as depicted in FIG. 3 in the gastrointestinal tract (e.g., esophagus), such the configuration would be able to fully dilate the tract muscle to keep tract tissues, particularly tissues at opposing positions, from coming into close contact. Note that it is essential to keep a cancerous tissue (e.g., esophageal tissue having tumor resides thereon) away from a normal tissue adjacent thereto during radiation treatment, so as to minimize unnecessary exposure of normal tissues to radioactive materials.

According to some embodiments of the present disclosure, the catheter further comprises a working conduit disposed adjacent to the plurality of communicating conduits. Referring to FIG. 4, which is the side sectional view of apparatus 20 along the lateral direction. The configuration of apparatus 20 is quite similar to that of apparatus 10, except the catheter 22 in this embodiment further comprises a working conduit 25, which is disposed in the center of the catheter 22, with two communicating conduits (24a and 24b; 24c and 24d) disposed on either sides. Note that the arrangement of the working conduit 25 and the communicating conduits 24a, 24b, 24c, 24d is for illustrative purpose only, and the scope of the present disclosure is not limited thereto. As would be appreciated, the arrangement of the working conduit and the communicating conduits may be modified in accordance with the practical needs.

The working conduit is configured to receive a medical instrument, an endoscope, a contrast agent, a radionuclide, a sensor or detector, or a shielding material. In general, the medical instrument may be any instrument or device that is used in a surgical procedure, for example, a biopsy needle, a needle, a tube, a cauterization device, a laser, a drill, a guidewire, a fiberoptic device, an electrode, a saw, an ultrasonic device, a spectroscopic device, an electrical sensor, a thermal sensor, a draining tube, or a combination thereof. The endoscope may be any instrument used to obtain a view of the interior of a patient's body via a variety of means to capture and transmit the view to an observer. The contrast agent is a substance used to increase the contrast of structures within the body; depending on desired purposes, the contrast agent may be a radiocontrast agent (e.g., iodine or barium), a magnetic resonance imaging (MRI) agent (e.g., gadolinium), or a ultrasound contrast agent (e.g., microbubble made of agitated saline solution, nitrogen, or perfluorocarbons). The radionuclide may be any of Barium-133, Cadmium-109, Cobalt-57, Cobalt-60, Europium-152, Manganese-54, Sodium-22, Zinc-65, Technetium-99m, Strontium-90, Thallium-204, Carbon-14, Tritium, Polonium-210, Uranium-238, Caesium-137, Americium-241, Iridium-77, Iridium-34, Iridium-192 or other active sources capable of emitting ionizing radiation. The sensor or detector is useful in measuring or detecting the physical condition of the subject, or the alteration of the catheter. Regarding the shielding material, it is configured to impede radiation emitted from a high energy source (e.g., EBRT); the shielding material may be made of a metal (e.g., barium, bismuth, tungsten, lead, aluminum, lithium, cadmium, gadolinium, or titanium), a metal alloy (e.g., a lead alloy, a titanium alloy, or a tungsten alloy), a polymer (e.g., polyisoprene, polybutadiene, styrene-butadiene, ethylene-propylene, silicone, polysulfide, or polyurethane), or a combination thereof. The front end of the working conduit may be an open end or a closed/blocked end.

Preferably, the catheter, communicating conduits, and/or working conduit of the present apparatus are independently made of a biocompatible material, for example, silicone, polyvinyl chloride, polyethylene, polypropylene, polyester, polyurethane, polyisobutylene, polychloroprene, polybutadiene, fibrin, collagen, gelatin, hyaluronan, polysaccharide, or a combination thereof. The catheter, communicating conduits, and/or working conduit of the present apparatus may be made from a single piece, or made from multiple pieces that are secured or butted together.

According to some embodiments, the catheter is no more than 20 mm in diameter; preferably, no more than 15 mm in diameter; more preferably, no more than 10 mm in diameter. In one specific embodiment, the diameter of the catheter is no more than 8 mm in diameter.

According to certain preferred embodiments, the fully inflated balloon is no more than 50 mm in diameter. More preferably, the fully inflated balloon is no more than 45 mm in diameter. According to one specific embodiment, the fully inflated balloon is no more than 40 mm in diameter.

Regarding the working conduit, it is about 0.5-20 mm in diameter; preferably, about 1-15 mm in diameter; more preferably, about 1-10 mm in diameter. In one specific embodiment, the diameter of the working conduit is about 1-5 mm in diameter.

Optionally, the present apparatus further comprises a movable or rotatable shielding material (e.g., a lead plate) disposed in and/or on the compliant balloon so as to adjust the treatment area or dosage of the radiation therapy administered to the subject.

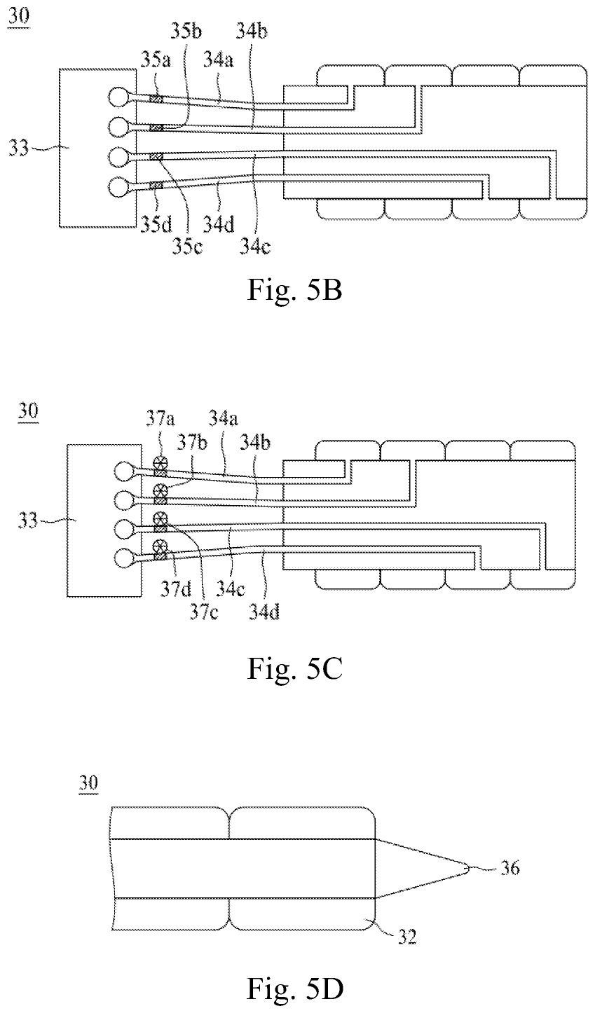

According to certain embodiments of the present disclosure, the present apparatus further comprises one or more fluid and/or air suppliers independently coupled to one or more of the communicating conduits. Referring to FIG. 5A, in which the apparatus 30 comprises a plurality of fluid and/or air suppliers 32a, 32b, 32c, 32d operably coupled to the plurality of the communicating conduits 34a, 34b, 34c, 34d. Note that the structure and/or arrangement of the compliant balloons, and the catheter are same as those in FIG. 4, thus are not repeated here for the sake of brevity. FIG. 5B illustrates an alternative configuration of the present apparatus, in which the apparatus 30 comprises one fluid and/or air supplier 33 operably coupled to the plurality of the communicating conduits 34a, 34b, 34c, 34d. The fluid and/or air supplier(s) is/are configured to independently provide a fluid or an air to the plurality of communicating conduits thereby independently controlling the inflating of each compliant balloon, which is in communications with the communicating conduits as described above.

Optionally, the apparatus 30 may further comprise a plurality of valves 35a, 35b, 35c, 35d respectively coupled to the plurality of the communicating conduits 34a, 34b, 34c, 34d (See, FIGS. 5A and 5B). The valves are configured to independently control the volume of the air or the fluid provided to each communicating conduit so as to alter the inflation volume of each compliant balloon.

Still optionally, the apparatus 30 may further comprise a plurality of indicators. Referring to FIG. 5C, in which the apparatus 30 comprises a plurality of indicators 37a, 37b, 37c, 37d respectively coupled to the plurality of the communicating conduits 34a, 34b, 34c, 34d. The indicators 37a, 37b, 37c, 37d are configured to independently indicate the volume of the air or the fluid provided from the fluid and/or air supplier 33 to each of the communicating conduits 34a, 34b, 34c, 34d. Depending on desired purposes, each of the indicator may be independently in the form of a pointer instrument or a balloon.

FIG. 5D illustrates an alternative configuration of the present apparatus, in which the apparatus 30 further comprises a cap 36 disposed at the front end of the catheter 32. In general, the cap may have a rounded end or a sharp end in accordance with intended uses. The configuration of the sharp end facilitates the insertion of the present apparatus into GI tract. According to optional embodiments of the present disclosure, the cap may have an agent (e.g., a contrast agent) contained therein.

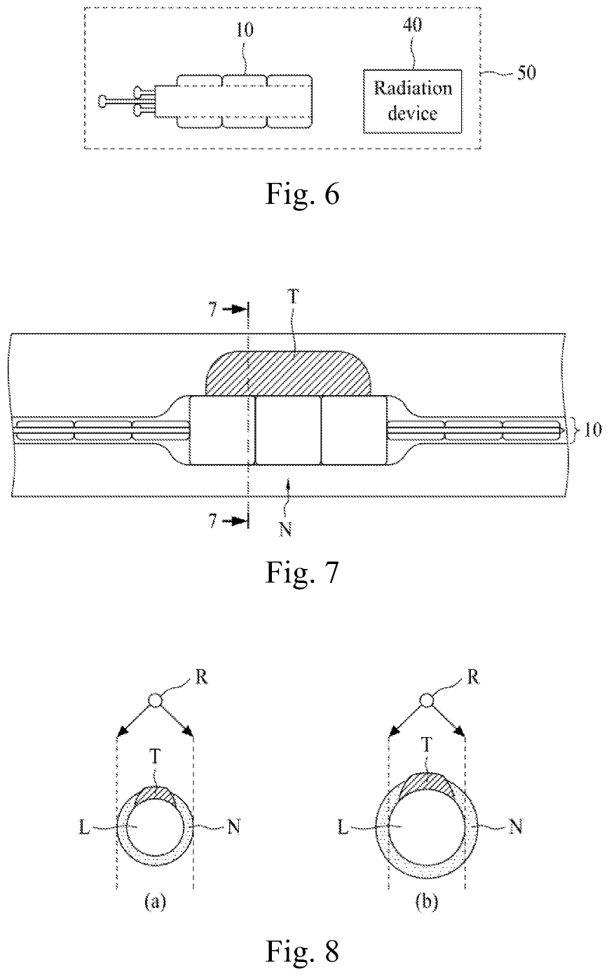

Another aspect of the present disclosure is directed to a radiotherapy system for treating a gastrointestinal tumor in a subject. Reference is now made to FIG. 6, which depicts a radiotherapy system 50 comprising an apparatus 10, and a radiation device 40. According to certain embodiments of the present disclosure, the apparatus 10 is configured to space the gastrointestinal tumor away from a normal tissue of the gastrointestinal tract of the subject, and the radiation device 40 is configured to provide an external beam radiation therapy to the gastrointestinal tumor.

The radiation device of the present disclosure may be any device suitable for delivering an external beam of radiation (e.g., a photon beam or a particle beam) to tumors for tumor-destroying purposes; examples of the radiation device include, but are not limited to, orthovoltage (superficial) X-ray machine, megavoltage X-ray machine, supervoltage X-ray machine, linear accelerator, cobalt unit, proton cyclotron, isochronous cyclotron, and synchrotron. Preferably, the radiation device of the present disclosure is a device for delivering a particle beam; more preferably, the radiation device is useful in executing PBT.

Also disclosed herein is a method of treating a gastrointestinal tumor in a subject with the aid of the present apparatus. Before starting treatment, the apparatus (e.g., the apparatus 10 of FIG. 1A) is inserted through the mouth or nose of the subject into the gastrointestinal tract of the subject. Then, depending on desired purposes, one or more compliant balloons (e.g., the complaint balloons 16a, 16b, 16c of apparatus 10) are inflated thereby spacing the gastrointestinal tumor away from normal tissues of the gastrointestinal tract of the subject. FIG. 7 is a schematic diagram illustrating the practical application of apparatus 10. Once entering the gastrointestinal tract, the clinical practitioner may dilate the tract muscle by controlling the inflation and/or deflation state of the compliant balloons 16a, 16b, 16c (e.g., increase or decrease respective volumes of the balloons) so that the gastrointestinal tumor (as denoted as “T” in FIG. 7) is completely spaced apart from normal gastrointestinal tissues (as denoted as “N” in FIG. 7) of the subject. In addition, the present apparatus may also be anchored in any desired position via controlling the inflation and/or deflation state of the compliant balloons 16a, 16b, 16c (e.g., increase or decrease respective volumes of the balloons).

FIG. 8 provides cross sectional views of the GI tract of FIG. 7, which views are taken as indicated by the section line 7-7 in FIG. 7. As illustrated in Panel (a) of FIG. 8, before inflating the compliant balloon (not shown in FIG. 8), normal gastrointestinal tissues (as denoted as “N” in FIG. 8) adjacent to the gastrointestinal tumor (as denoted as “T” in FIG. 8) is within the treatment area of radiation therapy. However, in the case when the lumen of GI tract is expanded by the present apparatus (not shown in FIG. 8), then the exposure of normal gastrointestinal tissues under radiation therapy would be greatly reduced thereby improving the accuracy of radiation therapy (Panel (b) of FIG. 8).

Then, an effective amount of EBRT is administered to the subject. The EBRT may be a photon beam radiation therapy (e.g., X-ray or gamma-ray therapy), or a particle therapy (e.g., proton, neutron or carbon ion therapy). According to preferred embodiments of the present disclosure, the EBRT is PBT. The protective effect of the present apparatus renders normal gastrointestinal tissues less susceptible to the EBRT thereby greatly reducing the side-effect of EBRT.

During the operation, the clinical practitioner may adjust the position of the apparatus in accordance with the size or distribution of the tumors, and the diameter or shape of GI tract via altering the volume of compliant balloons (e.g., the complaint balloons 16a, 16b, 16c of apparatus 10) to optimize the treatment of the gastrointestinal tumor.

Optionally, a radiation treatment planning is performed before the administration of EBRT, and the EBRT is administered in accordance with the radiation treatment planning.

The gastrointestinal tumor is any of an esophageal tumor, a stomach tumor, a tumor of bile duct, a gallbladder tumor, a pancreatic tumor, a small intestinal tumor, a colon tumor, a rectal tumor, or an anal tumor. According to certain embodiments of the present disclosure, the gastrointestinal tumor is esophageal tumor.

Alternatively, the present apparatus and/or method may be used to treat an aerodigestive tract tumor, i.e., the tumor of the respiratory tract, and the tumor of the upper part of the digestive tract. Exemplary aerodigestive tract tumors include, but are not limited to, the tumors of nasal cavity, paranasal sinuses, nasopharynx, oral cavity, oropharynx, larynx, hypopharynx, and part of the esophagus and windpipe.

The subject treatable with the present apparatus and/or method is a mammal, for example, a rat, a hamster, a guinea pig, a rabbit, a dog, a cat, a cow, a goat, a sheep, a monkey, and a horse. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

Materials and Methods

The applicator (i.e., the apparatus with 8 compliant balloons extended along the axial direction thereof) was placed into the gastrointestinal tract of the chest phantom. Computerized tomography (CT) scans were performed with the applicator before and after balloon inflation (having a diameter of 20 mm in an inflating state). The CT images were imported to the treatment planning system (TPS) RAYSTATION® for delineation of target volume and organ at risk (OAR), and the dose volume histogram (DVH) of proton pencil beam scanning (PBS) plans was analyzed.

### Example 1 Protective Effect of the Present Applicator in OAR

The total prescribed dose was 50Gy (relative biological effectiveness (RBE)), and the only planning objective was to deliver at least 95% of the prescription dose to at least 98% of the planning target volume (PTV). For each CT scan of the phantom with the applicator inflated or non-inflated, a PBS plan with three coplanar beams was optimized on the average intensity CT using robust optimization in TPS. The gantry angles, table angles, beam energies, number of layers, and monitor units of each plan were similar.

The volume of the lung receiving a dose of 5 Gy, 10 Gy, or 20 Gy (i.e., V5, V10, or V20) was calculated as an absolute volume and as a percentage of the total lung volume; the results were summarized in Table 1. Meanwhile, the volume of the esophagus receiving a dose of 5 Gy, 10 Gy, 20 Gy, 30 Gy, or 40 Gy (i.e., V5, V10, V20, V30, or V40) was also calculated as an absolute volume and as a percentage of the total esophagus volume; the results were summarized in Table 2.

TABLE 1
Protective effect on lung

| Dose | w/o Inflation | w/Inflation |
|---|---|---|
| 20 Gy | 6.82% | 5.0% |
| 10 Gy | 17.0% | 3.33% |
| 5 Gy | 24.54% | 7.92% |

TABLE 2
Protective effect on esophagus

| Tissue | Dose | w/o Inflation | w/Inflation |
|---|---|---|---|
| Esophagus-surface | 40 Gy | 46.03% | 39.11% |
| | 30 Gy | 53.7% | 45.48% |
| | 20 Gy | 61.0% | 51.14% |
| | 10 Gy | 68.94% | 58.06% |
| | 5 Gy | 75.14% | 63.5% |
| Esophagus | 40 Gy | 63.92% | 55.48% |
| | 30 Gy | 72.75% | 64.53% |
| | 20 Gy | 80.85% | 72.02% |
| | 10 Gy | 87.5% | 79.18% |
| | 5 Gy | 90.83% | 84.5% |

The phantom treated with the aid of the present balloon-inflated applicator demonstrated the reduction in radiation exposure of normal tissues during PBT, as compared with the control phantom treated without the aid of balloon inflation.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. An apparatus for use with an external beam radiotherapy (EBRT) to treat a gastrointestinal tumor in a subject, comprising,
a catheter, which comprises,
a plurality of communicating conduits; and
a plurality of compliant balloons extended outside and along the axial direction of the catheter, wherein,
each of the plurality of communicating conduits is in air or fluid communication with at least one of the plurality of compliant balloons; and
when the apparatus enters the gastrointestinal tract of a subject, each of the plurality of compliant balloons is configured to inflate in the axial and radial directions of the compliant balloon thereby conforming to the shape of the gastrointestinal tract of the subject, wherein the inflation in the axial direction ensures that there is substantially no dead space present between two adjacent balloons.

2. The apparatus of claim 1, wherein each of the plurality of compliant balloons independently comprises a supporting structure disposed inside the compliant balloon.

3. The apparatus of claim 1, wherein
each of the plurality of compliant balloon is in juxtaposition to its adjacent balloon; and
each of the plurality of compliant balloon has a central portion along the axial direction thereof, and a radial portion extending radially outward from the central portion, wherein the axial length of the central portion is equal to or less than the maximum axial length of the radial portion.

4. The apparatus of claim 1, wherein the catheter further comprises a working conduit disposed adjacent to the plurality of communicating conduits, wherein the working conduit is configured to receive a medical instrument, an endoscope, a contrast agent, a radionuclide, or a shielding material.

5. The apparatus of claim 1, wherein the apparatus comprises at least three communicating conduits and at least three compliant balloons, and each communicating conduit is in air or fluid communication with each compliant balloon.

6. The apparatus of claim 1, wherein each compliant balloon has two terminal portions and an intermediate portion disposed therebetween, and the intermediate portion is relatively thicker than each terminal portion.

7. The apparatus of claim 1, wherein the EBRT is particle therapy, wherein the particle therapy is proton beam therapy (PBT).

8. (canceled)

9. The apparatus of claim 1, wherein the gastrointestinal tumor is esophageal tumor.

10. The apparatus of claim 1, further comprising a fluid and/or air supplier operably coupled to the plurality of the communicating conduits, and configured to provide a fluid or an air to the plurality of communicating conduits.

11. The apparatus of claim 1, further comprising a plurality of fluid and/or air suppliers operably coupled to the plurality of the communicating conduits, and configured to independently provide a fluid or an air to the plurality of communicating conduits.

12. The apparatus of claim 10, further comprising a plurality of valves respectively coupled to the plurality of the communicating conduits, and each valve is configured to independently control the volume of the air or the fluid provided to each communicating conduit so as to alter the inflation volume of each compliant balloon.

13. The apparatus of claim 10, further comprising a plurality of indicators respectively coupled to the plurality of the communicating conduits, and each indicator is configured to independently indicate the volume of the air or the fluid provided to each communicating conduit.

14. The apparatus of claim 1, further comprising a cap disposed at the front end of the catheter.

15. (canceled)

16. A radiotherapy system for treating a gastrointestinal tumor in a subject, comprising the apparatus of claim 1, and a radiation device for use with the apparatus, wherein the apparatus is configured to space the gastrointestinal tumor away from a normal tissue of the gastrointestinal tract of the subject, and the radiation device is configured to provide an external beam radiation therapy (EBRT) to the gastrointestinal tumor.

17. A method of treating a gastrointestinal tumor in a subject with the aid of the apparatus of claim 1, comprising,
(a) inserting the apparatus through the mouth or nose of the subject into the gastrointestinal tract of the subject;
(b) inflating at least one of the compliant balloons so as to space the gastrointestinal tumor away from a normal tissue of the gastrointestinal tract of the subject;
(c) administering to the gastrointestinal tumor an effective amount of EBRT; and
(d) optionally, adjusting the position of the apparatus via altering the inflation volume of at least one of the compliant balloons to optimize the treatment of the gastrointestinal tumor.

18. The method of claim 17, wherein the EBRT is particle therapy.

19. The method of claim 18, wherein the particle therapy is proton beam therapy (PBT).

20. The method of claim 17, wherein the gastrointestinal tumor is esophageal tumor.

21. The apparatus of claim 11, further comprising a plurality of valves respectively coupled to the plurality of the communicating conduits, and each valve is configured to independently control the volume of the air or the fluid provided to each communicating conduit so as to alter the inflation volume of each compliant balloon.

22. The apparatus of claim 11, further comprising a plurality of indicators respectively coupled to the plurality of the communicating conduits, and each indicator is configured to independently indicate the volume of the air or the fluid provided to each communicating conduit.
